(19) United States
(12) Patent Application Publication
(10) Pub. No.: US 2003/0068283 A1
(43) Pub. Date: Apr. 10, 2003

(54) Composition

(75) Inventors: Victoria Cromwell, Merseyside (GB); Peter Freunscht, Merseyside (GB); Peter John Hall, Merseyside (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/225,856

(22) Filed: Aug. 22, 2002

(30) Foreign Application Priority Data
Aug. 24, 2001 (EP) 01307269.9
Dec. 11, 2001 (EP) 01310338.7

(51) Int. Cl. A61K 7/16

(52) U.S. Cl. 424/49

(57) ABSTRACT

Topical composition comprising an alkyl hydroxybenzoate represented by formula I

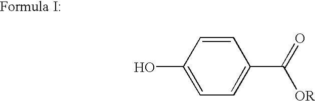

wherein R represents an alkyl group comprising at least five carbon atoms, characterised in that the composition additionally comprises a divalent metal salt.

### INTRODUCTION

Alkyl hydroxybenzoates (parabens) are known in the art where the alkyl group is methyl. For example, methyl hydroxybenzoate is mentioned, albeit fleetingly, for use in medicinal and oral care preparations as a preservative (WO 00/09507 and WO 00/69401).

In addition, U.S. Pat. No. 5,094,841 (Fine) discloses the use of heptyl paraben as a preservative in an oral care formulation. However, it also states that the preferred preservatives are methyl and propyl paraben and only ever states that they may be included in small amounts (0.1%) to provide a preservative effect.

We have found that there exists a range of compounds which exhibit surprisingly high antibacterial efficacy and are not disclosed as such in the prior art. Further, there exists a synergistic effect between these compounds and divalent metal ions.

### STATEMENT OF INVENTION

Accordingly, the invention provides an oral composition comprising an alkyl hydroxybenzoate represented by formula I

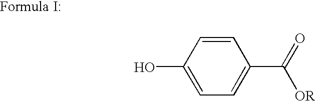

wherein R represents an alkyl group comprising at least five carbon atoms, characterised in that the composition additionally comprises a divalent metal salt.

### DESCRIPTION OF INVENTION

The alkyl group of the compound according to formula 1 is an alkyl comprising more than five carbon atoms. Preferably, the alkyl group comprises no more than 30 carbon atoms. More preferably the alkyl group comprises from 6 to 15 carbon atoms, especially from 6 to 10 and especially preferably, from 7 to 8.

Further, the alkyl group may be branched or straight chain and/or substituted or unsubstituted.

Preferred alkyl groups include octyl, heptyl and 2-ethylhexyl, more preferably, n-octyl or 2-ethylhexyl. Such compounds may be made by simple esterification of 4-hydroxybenzoic acid with the respective alcohol. Such a process is a simple step for the man skilled in the art to carry out to a reasonable degree.

The compound according to formula 1 is preferably present in an amount such that an antibacterial effect can be provided. In practice this ranges from 0.15 to 30% by weight of the composition according to the invention. Preferably, in an amount ranging from 0.2 to 20% by weight and even more suitably from 0.25 to 5% by weight. In a most preferred embodiment the paraben comprises from 0.5 to 2.5% by weight of the composition.

The composition according to the invention also comprises a divalent metal salt. Preferably, the divalent metal salt is a salt selected from the group consisting of zinc- and stannous salts such as zinc citrate, zinc sulphate, zinc glycinate, sodium zinc citrate, stannous pyrophosphate and mixtures thereof. The preferable divalent metal salt is zinc citrate.

Suitably, the amount of divalent metal salt ranges from 0.01 to 10% by weight of the composition, preferably from 0.05 to 5% by weight, more preferably from 0.1 to 2% by weight and especially preferably from 0.3 to 0.9% by weight of the composition.

In a second aspect the invention provides the use of a synergistic combination of a divalent metal salt with a parahydroxy benzoic acid as herein described for the treatment selected from the group consisting of anti-plaque, anti-caries, anti-gingivitis, anti-tartar, anti-malodour and combinations thereof.

In a third aspect the invention provides the use of a synergistic combination of a divalent metal salt with a parahydroxy benzoic acid as herein described in the manufacture of a medicament for the treatment selected from the group consisting of anti-plaque, anti-caries, anti-gingivitis, anti-tartar, anti-malodour and combinations thereof.

The oral composition according to the invention comprise further ingredients which are common in the art, such as:

antimicrobial agents, e.g. Triclosan, chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol); anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E; plant extracts; desensitising agents, e.g. potassium citrate, potassium chloride, potassium tartrate, potassium bicarbonate, potassium oxalate, potassium nitrate and strontium salts; anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants; particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickeners such as sodium carboxymethyl-cellulose, xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

The oral compositions may be in any form common in the art, e.g. toothpaste, gel, mousse, aerosol, gum, lozenge, powder, cream, etc. and may also be formulated into systems for use in dual-compartment type dispensers.

Embodiments according to the invention shall now be discussed with reference to the following non-limiting examples.

### EXAMPLE

The following is a formulation according to the present invention. It is made by known processes.

| Ingredient | % w/w |
|---|---|
| 70% aq. sorbitol | 45.0 |
| Saccharin | 0.2 |
| Polyethylene glycol | 2.0 |
| Titanium dioxide | 1.0 |
| Sodium fluoride | 0.32 |
| Thickening silica | 9.0 |
| Abrasive silica | 10.0 |
| SLS | 1.6 |
| Sodium carboxymethylcellulose | 0.8 |
| Flavour | 1.0 |
| Zinc citrate trihydrate | 0.75 |
| n-Octyl paraben | 1.0 |
| Water | to 100 |

1. Oral composition comprising an alkyl hydroxybenzoate represented by formula I 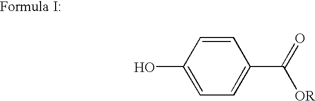
wherein r represents an alkyl group comprising at least five carbon atoms, characterised in that the composition additionally comprises a divalent metal salt:

2. Composition according to claim 1, wherein R represents an alkyl group comprising from six to fifteen carbon atoms.

3. Composition according to claim 1, wherein R represents an alkyl group comprising from seven to ten carbon atoms.

4. Composition according to claim 1, wherein R represents a group selected from the group consisting of octyl and heptyl.

5. Composition according to any of claim 1, wherein R represents a branched alkyl group.

6. Composition according to claims 1, wherein R is 2-ethylhexyl.

7. Composition according to claim 1, wherein the composition is an oral composition and comprises an orally acceptable carrier.

8. Composition according to claim 1, wherein the composition is a composition selected from the group consisting of paste, gel, foam, liquid, powder, chewing gum and is suitable for use in dental care.

9. Composition according to claim 1, wherein the composition comprises from 0.1 to 10% by weight of the alkyl hydroxybenzoate.

10. Composition according to claim 1, wherein the divalent metal salt is a salt selected from the group consisting of zinc citrate, zinc sulphate, zinc fluoride, zinc glycinate, stannous citrate and mixtures thereof.

11. Composition according to claim 1, wherein the divalent metal salt is present in an amount ranging from 0.01 to 10% by weight of the composition.

12. Composition according to claim 1, wherein the divalent metal salt is zinc citrate and is present in an amount ranging from 0.01 to 5% by weight of the composition.

13. A method of treating a condition selected from the group consisting of bad breath, caries, tartar, gingivitis and tooth stains by applying to the teeth an oral composition comprising a synergistic combination of a divalent metal salt and a parahydroxy benzoic acid.
